(19) United States
(12) Patent Application Publication
(10) Pub. No.: US 2025/0290887 A1
(43) Pub. Date: Sep. 18, 2025

(54) GAS SENSOR

(71) Applicant: TDK Corporation, Tokyo (JP)

(72) Inventors: Makoto SHIBATA, Tokyo (JP); Takumi MATSUO, Tokyo (JP)

(73) Assignee: TDK Corporation, Tokyo (JP)

(21) Appl. No.: 19/075,904

(22) Filed: Mar. 11, 2025

(30) Foreign Application Priority Data
Mar. 14, 2024 (JP) 2024-040278

(51) Int. Cl. G01N 27/18; G01N 33/00

(52) U.S. Cl. CPC G01N 27/18; G01N 33/0027

(57) ABSTRACT

A gas sensor includes a substrate with an opening, an insulating film disposed at a location overlapping the opening in plan view, and a gas detecting unit on an upper surface of the insulating film. The insulating film is configured to have a warped shape protruding upwards during gas detection, in which the gas detecting unit can detect a gas.

### CROSS-REFERENCE TO RELATED APPLICATIONS

The present application claims a priority to Japanese patent application No. 2024-040278 filed on Mar. 14, 2024, which is incorporated herein by reference in its entirety.

### BACKGROUND

The present disclosure relates to a gas sensor.

A gas sensor is a device that detects a gas present in an atmosphere and converts information (e.g., type and concentration) of the gas into an electrical signal for output. Such a gas sensor is included in home appliances, industrial equipment, environmental monitoring apparatuses, and the like and is used for detecting the concentration of a specific gas affecting humanity, the environment, and the like.

Disclosed as a gas sensor is a gas sensor including a detecting unit disposed on a flat insulating film disposed at an opening portion of a substrate (see, for example, Patent Document 1).

Patent Document 1: JP Patent No. 5100733 Specification

### SUMMARY

A gas sensor according to the present disclosure includes
a substrate with an opening,
an insulating film disposed at a location overlapping the opening in plan view, and
a gas detecting unit on an upper surface of the insulating film,
wherein the insulating film is configured to have a warped shape protruding upwards during gas detection, in which the gas detecting unit can detect a gas.

### BRIEF DESCRIPTION OF THE DRAWING(S)

FIG. 1 is a schematic plan view of a gas sensor according to a first embodiment of the present disclosure.

FIG. 2 is a schematic sectional view of the gas sensor shown in FIG. 1 along a line II-II.

FIG. 3 is a schematic sectional view of the gas sensor shown in FIG. 1 along a line III-III.

FIG. 4 is a schematic sectional view of the gas sensor shown in FIG. 1 along a line IV-IV.

FIG. 5 is an enlarged sectional view of an insulating film and a gas detecting unit of the gas sensor shown in FIG. 1 in a state before gas detection.

FIG. 6 is a view of the insulating film and the gas detecting unit shown in FIG. 5 in a state during gas detection.

FIG. 7 is a plan view of a shape of a substrate of the gas sensor shown in FIG. 1.

FIG. 8A and FIG. 8B are plan views of a shape of a third insulating film of the gas sensor shown in FIG. 1.

FIG. 9A and FIG. 9B are plan views of a shape of a second heater wire of the gas sensor shown in FIG. 1.

FIG. 10A and FIG. 10B are plan views of a shape of a second insulating film of the gas sensor shown in FIG. 1.

FIG. 11A and FIG. 11B are plan views of a shape of a first heater wire of the gas sensor shown in FIG. 1.

FIG. 12A and FIG. 12B are plan views of a shape of a first insulating film of the gas sensor shown in FIG. 1.

FIG. 13A and FIG. 13B are plan views of shapes of detecting electrodes of the gas sensor shown in FIG. 1.

FIG. 14A and FIG. 14B are plan views of a shape of a thermistor film of the gas sensor shown in FIG. 1.

FIG. 15 is a schematic view of a shape of the gas sensor according to the first embodiment during gas detection and a gas flow near the gas sensor.

FIG. 16A and FIG. 16B are plan views of a shape of a gas detecting unit of a gas sensor according to a second embodiment of the present disclosure.

FIG. 17A is a plan view of a shape of a heater wire and an insulating film of a gas sensor according to a third embodiment of the present disclosure; and FIG. 17B is an enlarged sectional view of the heater wire, the insulating film, and the like shown in FIG. 17A.

FIG. 18A is a plan view of shapes of heater wires and an insulating film of a gas sensor according to a fourth embodiment of the present disclosure; and FIG. 18B is an enlarged sectional view of the heater wires and the insulating film shown in FIG. 18A.

FIG. 19A is a plan view of shapes of heater wires and an insulating film of a gas sensor according to a fifth embodiment of the present disclosure; and FIG. 19B is an enlarged sectional view of the heater wires and the insulating film shown in FIG. 19A.

### DETAILED DESCRIPTION

A conventional gas sensor, in which its detecting unit is disposed on a flat insulating film, may not have enough chances of the detecting unit coming into contact with a gas flow. For example, in a situation where a gas flow parallel to the flat insulating film is generated near the gas sensor, the area of the detecting unit with chances of coming into contact with the gas flow is limited, which may unfortunately impair, for example, high-speed responsivity.

It is desirable to provide a gas sensor that can have better chances of a gas detecting unit coming into contact with a gas flow and has suitable responsiveness.

Hereinafter, the present disclosure is described in detail in the following order with reference to specific embodiments or the like.
1. First embodiment
1.1. Overall structure
1.2. Substrate
1.3. Insulating film
1.4. Heater wires
1.5. Gas detecting unit
1.6. Manufacturing method
1.7. Operation
2. Second embodiment
3. Third embodiment
4. Fourth embodiment
5. Fifth embodiment

### 1. First Embodiment

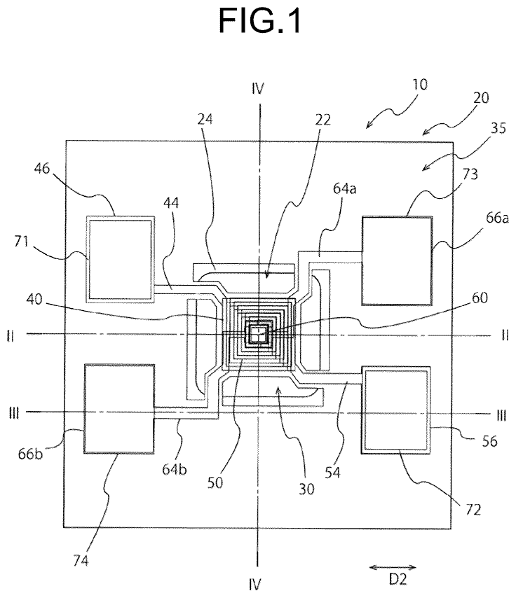

FIG. 1 is a schematic plan view of a gas sensor 10 according to a first embodiment viewed from above. Note that FIG. 1 shows, among members included in the gas sensor 10, only a substrate 20 and a third insulating film 33 in a nontransparent manner and other members with their outlines overlapping so that shapes of members underneath are visible.

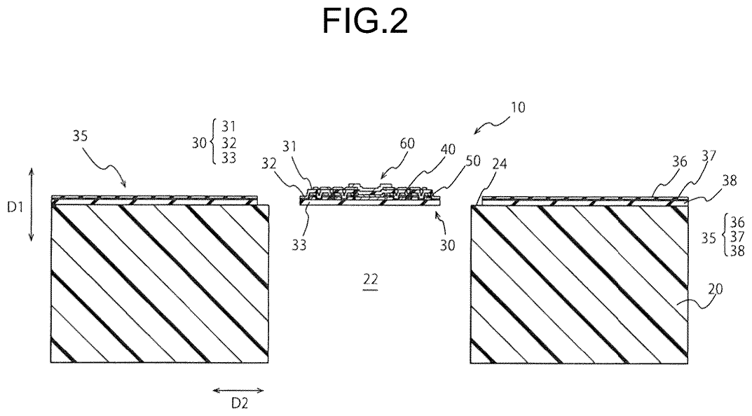

FIG. 2 is a sectional view of the gas sensor 10 shown in FIG. 1 along a section line II-II. As shown in FIGS. 1 and 2, the gas sensor 10 includes the substrate 20 with an opening 22, an insulating film 30 disposed at a location overlapping the opening 22 in plan view from a direction perpendicular to a substrate upper surface 24, and a gas detecting unit 60 on an upper surface of the insulating film 30. Examples of gases of interest detectable by the gas detecting unit 60 include carbon dioxide (CO₂).

As described later in detail, the gas detecting unit 60 of the gas sensor 10 includes a thermistor film 61 whose resistance changes by contact with a gas of interest. Changes in the concentration of the gas of interest causes changes in thermal conductivity of the gas in contact with the thermistor film 61, thus changing the temperature of the thermistor film 61 to change its resistance. The gas detecting unit 60 also includes at least a pair of electrode portions 62a and 62b in contact with the thermistor film 61. Using changes in resistance of the thermistor film 61 connecting the electrode portions 62a and 62b, the gas sensor 10 detects the concentration of the gas.

Note that the gas detecting unit 60 included in a gas sensor according to the present disclosure is not limited to the gas detecting unit including the thermistor film 61 and the pair of electrode portions 62a and 62b; and examples of gas detecting units include a gas detecting unit with, instead of the thermistor film 61, a thin MOx (metal oxide) film (see, for example, Second embodiment). In the gas sensor 10, the portion that comes into contact with a gas and reacts may be in a thin film shape. However, gas sensors according to the present disclosure include those with an optional gas detecting unit, such as a gas sensor with a referential element that does not have a direct gas detecting function and a gas sensor with different gas detecting units. Other than that, as examples of the gas detecting unit 60 included in a gas sensor according to the present disclosure, a thermal conduction type gas detecting unit, in which a Pt wire is used instead of a thermistor film and electrodes, and a catalytic combustion type gas detecting unit, in which a catalyst and electrodes are used, may be mentioned. Examples of catalysts include noble metal particles (e.g., platinum (Pt), palladium (Pd), ruthenium (Ru), and rhodium (Rh)) supported on a support of an oxide material (e.g., aluminum oxide (e.g., γ-alumina) and silicon oxide).

### 1.1. Overall Structure

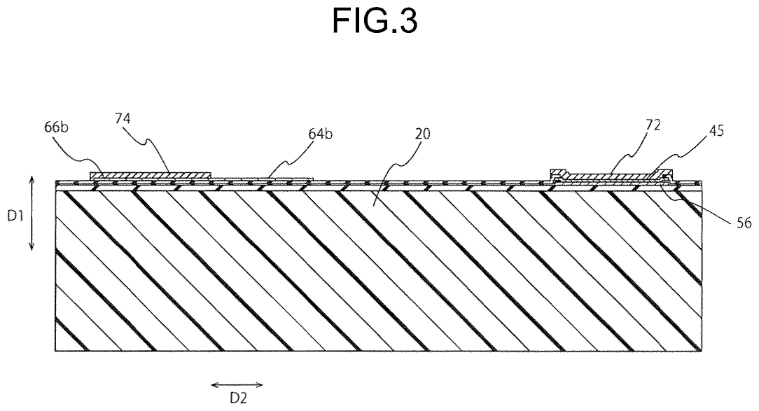

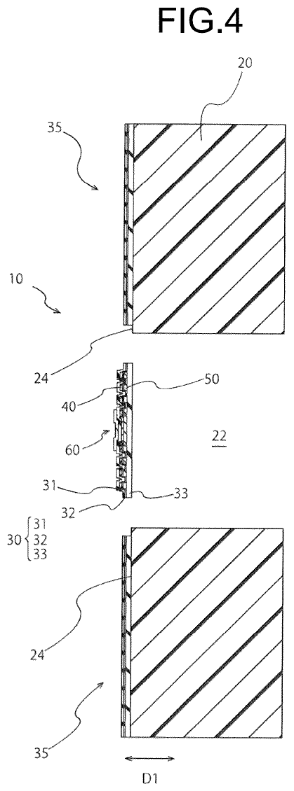

FIG. 3 is a schematic sectional view of the gas sensor 10 cut in a plane along a line III-III shown in FIG. 1. FIG. 4 is a schematic sectional view of the gas sensor 10 cut in a plane along a line IV-IV shown in FIG. 1. In the description of the gas sensor 10, the direction perpendicular to the substrate upper surface 24 is referred to as a vertical direction D1, and a direction parallel to the substrate upper surface 24 is referred to as a film surface direction D2. With regard to the vertical direction D1, a direction or a side away from the substrate 20 with respect to the substrate upper surface 24, where an insulating film peripheral portion 35 is provided, is referred to as an upward direction (upwards), an upper side, or being on or above something else; and a direction or a side towards the inside of the substrate 20 with respect to the substrate upper surface 24 is referred to as a downward direction (downwards), a lower side, or being below, under, or underneath something else.

As shown in FIGS. 1 and 2, the gas sensor 10 includes the substrate 20 with the opening 22. Other members of the gas sensor 10 are disposed at locations overlapping the opening 22 of the substrate 20 in plan view and over the substrate upper surface 24 around the opening 22. The members other than the substrate 20 included in the gas sensor 10 include the insulating film 30, a first heater wire 40, a second heater wire 50, and the gas detecting unit 60, which are disposed at the locations overlapping the opening 22 in plan view; and the insulating film peripheral portion 35, a first heater terminal portion 46, a second heater terminal portion 56, and electrode terminal portions 66a and 66b, which are disposed over the substrate upper surface 24.

### 1.2. Substrate

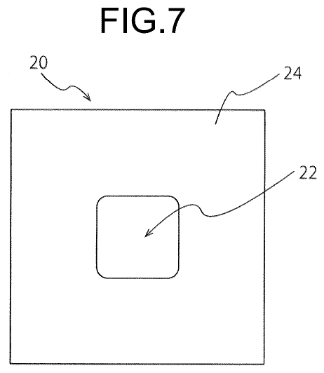

FIG. 7 is a plan view of a shape of the substrate 20 of the gas sensor 10. As shown in FIGS. 1 and 7, the opening 22 is provided near a center of the substrate 20. As shown in FIG. 2, in a region at an upper side of the opening 22, the insulating film 30 and the like are disposed. As shown in FIGS. 1 and 2, viewed from the vertical direction D1, the insulating film 30 is disposed so as to overlap the opening 22.

As shown in FIGS. 1, 2, and 7, the opening 22 of the substrate 20 constitutes a through-hole having a substantially rectangular shape in plan view; however, the shape of the opening 22 in plan view is not limited to the rectangular shape. Also, the opening 22 is not limited to the through-hole shown in FIGS. 2 and 4 and may constitute a recess that is recessed downwards from the substrate upper surface 24.

The substrate 20 may be made from any material that has mechanical strength to the extent that the members at the locations overlapping the opening 22 in plan view and over the substrate upper surface 24 can be supported and that is suitable for microfabrication (e.g., etching). In the present embodiment, examples of the substrate 20 include a silicon single crystal substrate, a sapphire single crystal substrate, a ceramic substrate, a quartz substrate, a glass substrate, and a ferrite substrate.

### 1.3. Insulating Film

As shown in FIGS. 1 and 2, in the gas sensor 10, the insulating film 30 is disposed at a location overlapping the opening 22 at a central portion of the substrate 20 in plan view. On the upper surface of the insulating film 30, the gas detecting unit 60 is provided. The insulating film 30 is a multilayer in which a first insulating film 31, a second insulating film 32, and the third insulating film 33 are stacked. Between the first insulating film 31 and the second insulating film 32 is the first heater wire 40. Between the second insulating film 32 and the third insulating film 33 is the second heater wire 50.

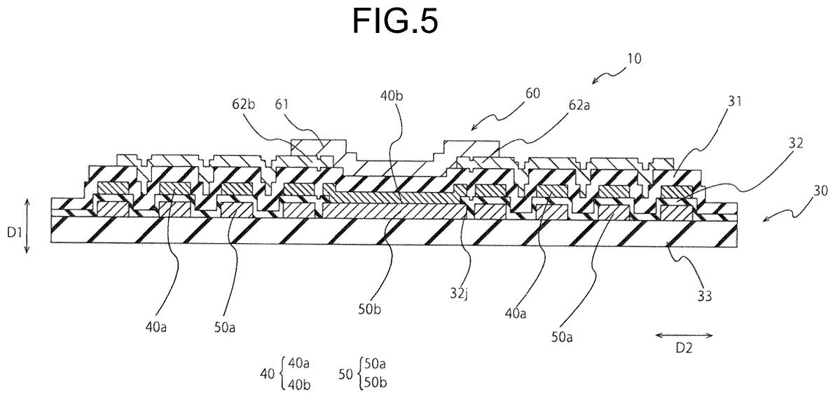

FIG. 5 is an enlarged sectional view of the gas detecting unit 60, the insulating film 30, the first heater wire 40, and the second heater wire 50 of the gas sensor 10 shown in FIG. 2. As shown in FIG. 5, the insulating film 30 includes the first insulating film 31, the second insulating film 32 below the first insulating film 31, and the third insulating film 33 below the second insulating film 32.

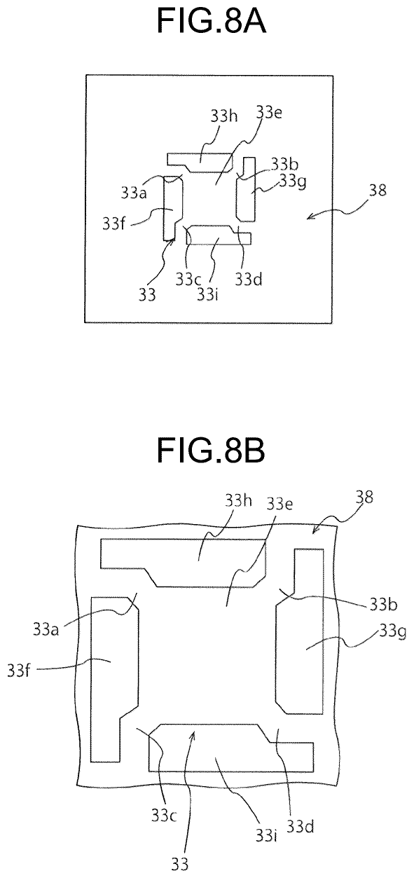

FIG. 8A is a plan view of the third insulating film 33 and a third insulating film peripheral portion 38, which is a part of a layer (film) constituting the third insulating film 33. FIG. 8B is a fragmentary enlarged view of the surroundings of the third insulating film 33 in FIG. 8A. Among the first insulating film 31, the second insulating film 32, and the third insulating film 33 constituting the insulating film 30, the third insulating film 33 is disposed at a lowest level. Also, as shown in FIGS. 2 to 4, the third insulating film peripheral portion 38 is disposed below a first insulating film peripheral portion 36 and a second insulating film peripheral portion 37.

As is understandable from comparison between FIGS. 8A and 1, the third insulating film 33 is disposed at a location overlapping the opening 22 of the substrate 20 in plan view, and the third insulating film peripheral portion 38 is provided on the substrate upper surface 24 of the substrate 20. The third insulating film 33 and the third insulating film peripheral portion 38 are simultaneously formed to integrally constitute one film. As shown in FIG. 2, a lower surface of the third insulating film peripheral portion 38 is in direct contact with the substrate upper surface 24.

As shown in FIG. 8B, the third insulating film 33 includes a central portion 33e in a substantially rectangular shape and a plurality of (in the present embodiment, four) beam portions 33a, 33b, 33c, and 33d connecting the central portion 33e and the third insulating film peripheral portion 38. The beam portions 33a, 33b, 33c, and 33d are provided at four corners of the central portion 33e. At portions of the central portion 33e other than the four corners, peripheral holes 33f, 33g, 33h, and 33i are provided so as to surround the central portion 33e. Arrangement of the third insulating film 33 at the location overlapping the opening 22 in plan view and the peripheral holes 33f, 33g, 33h, and 33i surrounding the central portion 33e can mitigate heat transfer between the third insulating film 33 and the third insulating film peripheral portion 38.

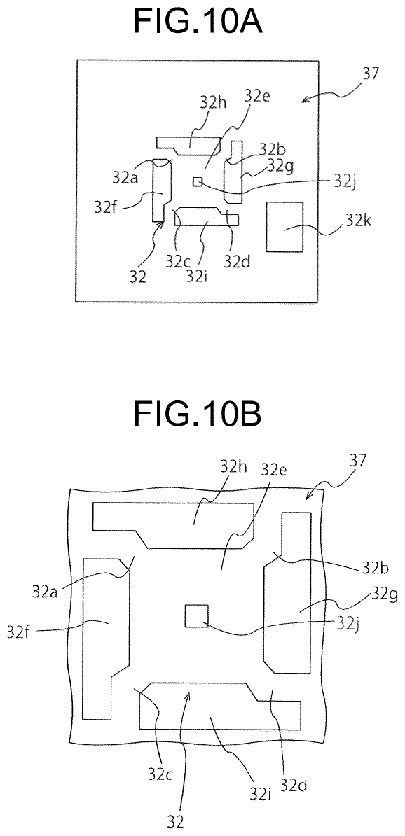

FIG. 10A is a plan view of the second insulating film 32 and the second insulating film peripheral portion 37, which is a part of a layer (film) constituting the second insulating film 32. FIG. 10B is a fragmentary enlarged view of the surroundings of the second insulating film 32 in FIG. 10A. Among the first insulating film 31, the second insulating film 32, and the third insulating film 33 constituting the insulating film 30, the second insulating film 32 is disposed between the first insulating film 31 and the third insulating film 33. Also, as shown in FIGS. 2 to 4, the second insulating film peripheral portion 37 is disposed between the first insulating film peripheral portion 36 and the third insulating film peripheral portion 38.

As is understandable from comparison between FIGS. 10A and 8A, the second insulating film 32 has a planar shape similar to that of the third insulating film 33 except that a central portion 32e of the second insulating film 32 has a heater connection hole 32j. The second insulating film peripheral portion 37 has a planar shape similar to that of the third insulating film peripheral portion 38 except for having a heater insertion hole 32k. As is understandable from comparison between FIGS. 10A and 1, the second insulating film 32 is disposed at a location overlapping the opening 22 of the substrate 20 in plan view, and the second insulating film peripheral portion 37 is provided above the substrate upper surface 24 of the substrate 20 and the third insulating film peripheral portion 38. The second insulating film 32 and the second insulating film peripheral portion 37 are simultaneously formed to integrally constitute one film. As shown in FIG. 2, a lower surface of the second insulating film peripheral portion 37 is in direct contact with an upper surface of the third insulating film peripheral portion 38.

As shown in FIG. 10B, the second insulating film 32 includes the central portion 32e in a substantially rectangular shape and a plurality of (in the present embodiment, four) beam portions 32a, 32b, 32c, and 32d connecting the central portion 32e and the second insulating film peripheral portion 37. The beam portions 32a, 32b, 32c, and 32d are provided at four corners of the central portion 32e. At portions of the central portion 32e other than the four corners, peripheral holes 32f, 32g, 32h, and 32i are provided so as to surround the central portion 32e. The peripheral holes 32f, 32g, 32h, and 32i surrounding the central portion 32e in this manner can mitigate heat transfer between the second insulating film 32 and the second insulating film peripheral portion 37.

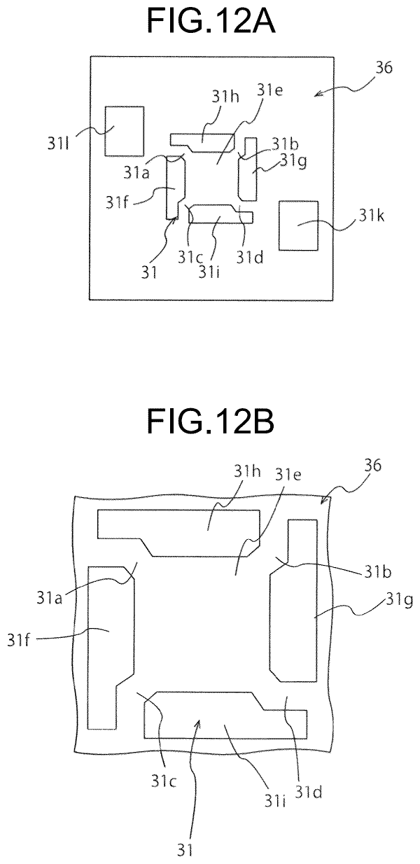

FIG. 12A is a plan view of the first insulating film 31 and the first insulating film peripheral portion 36, which is a part of a layer (film) constituting the first insulating film 31. FIG. 12B is a fragmentary enlarged view of the surroundings of the first insulating film 31 in FIG. 12A. Among the first insulating film 31, the second insulating film 32, and the third insulating film 33 constituting the insulating film 30, the first insulating film 31 is disposed at an uppermost level. Also, as shown in FIGS. 2 to 4, the first insulating film peripheral portion 36 is disposed above the second insulating film peripheral portion 37 and the third insulating film peripheral portion 38.

As is understandable from comparison between FIGS. 12A and 8A, the first insulating film 31 has a planar shape similar to that of the third insulating film 33. Also, the first insulating film peripheral portion 36 has a planar shape similar to that of the third insulating film peripheral portion 38 except for having heater insertion holes 31k and 31l. As is understandable from comparison between FIGS. 12A and 1, the first insulating film 31 is disposed at a location overlapping the opening 22 of the substrate 20 in plan view, and the first insulating film peripheral portion 36 is provided above the substrate upper surface 24 of the substrate 20, the third insulating film peripheral portion 38, and the second insulating film peripheral portion 37. The first insulating film 31 and the first insulating film peripheral portion 36 are simultaneously formed to integrally constitute one film. As shown in FIG. 2, a lower surface of the first insulating film peripheral portion 36 is in direct contact with an upper surface of the second insulating film peripheral portion 37.

As shown in FIG. 12B, the first insulating film 31 includes a central portion 31e in a substantially rectangular shape and a plurality of (in the present embodiment, four) beam portions 31a, 31b, 31c, and 31d connecting the central portion 31e and the first insulating film peripheral portion 36. The beam portions 31a, 31b, 31c, and 31d are provided at four corners of the central portion 31e. At portions of the central portion 31e other than the four corners, peripheral holes 31f, 31g, 31h, and 31i are provided so as to surround the central portion 31e. The peripheral holes 31f, 31g, 31h, and 31i surrounding the central portion 31e in this manner can mitigate heat transfer between the first insulating film 31 and the first insulating film peripheral portion 36.

As described above, the insulating film 30 is the multilayer including the first insulating film 31, the second insulating film 32, and the third insulating film 33. As shown in FIGS. 1, 8A, 10A, and 12A, the insulating film 30 is connected, via the beam portions 31a to 31d, 32a to 32d, and 33a to 33d of the first to third insulating films 31 to 33, to the first to third insulating film peripheral portions 36 to 38 provided above the substrate upper surface 24. The insulating film 30 is thus supported by its beam portions 31a to 31d, 32a to 32d, and 33a to 33d with respect to the first to third insulating film peripheral portions 36 to 38 and the substrate 20. This holds the insulating film 30 at the location overlapping the opening 22 in plan view as shown in FIG. 2.

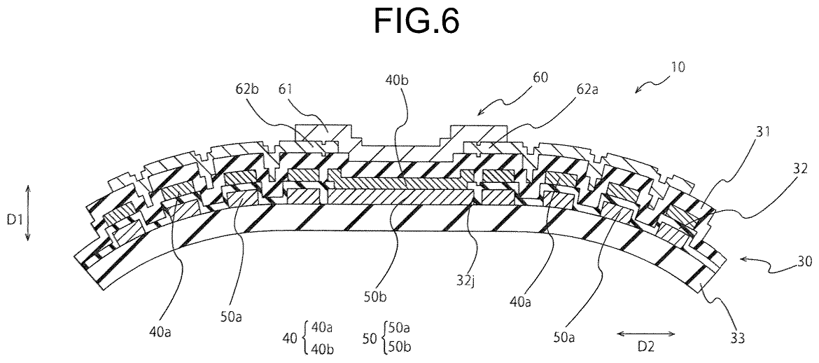

As shown in FIG. 5, the first insulating film 31, the second insulating film 32, and the third insulating film 33 may have different thicknesses. In a situation where the first insulating film 31, the second insulating film 32, and the third insulating film 33 have different thicknesses, the thickness of the third insulating film 33 being larger than the thickness of the first insulating film 31 and the thickness of the second insulating film 32 may ensure strength of the gas sensor 10 and deform the insulating film 30 in a warped shape protruding upwards as shown in FIG. 6. The thickness of the second insulating film 32 being smaller than the thickness of the third insulating film 33 and the thickness of the first insulating film 31 may shorten a step generated at a flat portion 40b, where the first heater wire 40 and the second heater wire 50 are connected. However, the first insulating film 31, the second insulating film 32, and the third insulating film 33 may have the same thickness.

The first insulating film 31, the second insulating film 32, and the third insulating film 33 may be made from any insulating film. Examples of such materials include SiO₂ (silicon oxide) and SiNx (silicon nitride).

### 1.4. Heater Wires

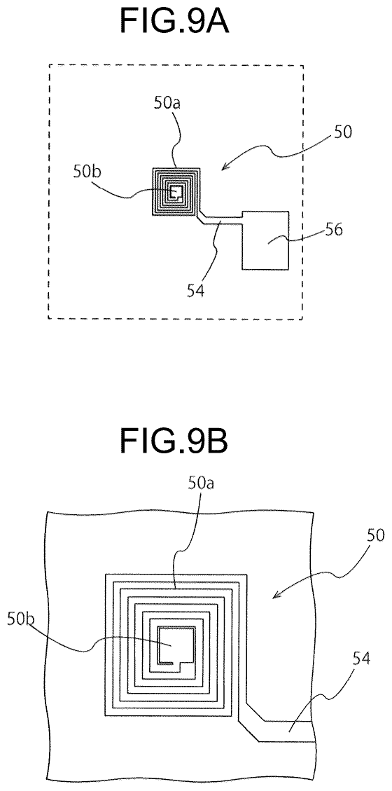

As shown in FIG. 5, the gas sensor 10 includes the first heater wire 40 in between the first insulating film 31 and the second insulating film 32 and the second heater wire 50 in between the second insulating film 32 and the third insulating film 33. FIG. 9A is a plan view of the second heater wire 50 and a second heater lead-out portion 54 and the second heater terminal portion 56, which are parts of a layer (film) constituting the second heater wire 50. FIG. 9B is a fragmentary enlarged view of the surroundings of the second heater wire 50 in FIG. 9A.

As is understandable from comparison between FIGS. 9A and 1, the second heater wire 50 is disposed at a location overlapping the opening 22 of the substrate 20 in plan view, and the second heater terminal portion 56 is provided above the substrate upper surface 24 of the substrate 20. The second heater lead-out portion 54 connects the second heater wire 50 and the second heater terminal portion 56. A part of the second heater lead-out portion 54 is disposed at a location overlapping the opening 22 in plan view and is in between the second insulating film 32 and the third insulating film 33. Another part of the second heater lead-out portion 54 is in between the second insulating film peripheral portion 37 (see FIG. 10A) and the third insulating film peripheral portion 38 (see FIG. 8A) above the substrate upper surface 24.

As shown in FIGS. 9B and 5, the second heater wire 50 includes a flat portion 50b, which is disposed at a central portion of the insulating film 30, and a heat-generating portion 50a in a spiral shape around the flat portion 50b. The heat-generating portion 50a has a smaller sectional area orthogonal to a conduction direction than the sectional area of the flat portion 50b or the second heater lead-out portion 54. Thus, with a flow of a current flowing the second heater wire 50 and the second heater lead-out portion 54, the heat-generating portion 50a effectively generates heat.

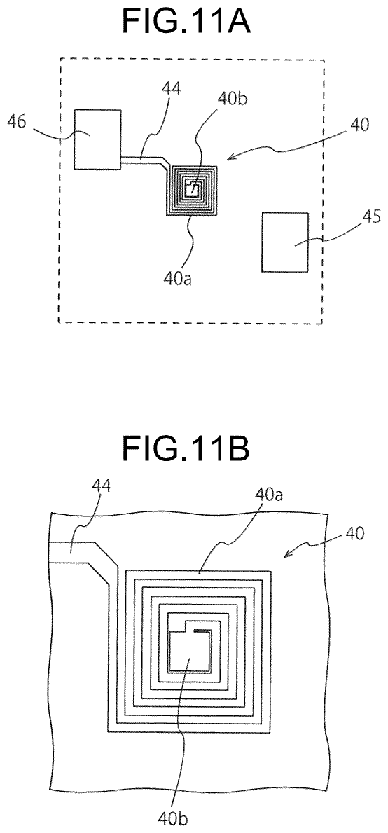

FIG. 11A is a plan view of the first heater wire 40 and a first heater lead-out portion 44, the first heater terminal portion 46, and a discontinuous portion 45, which are parts of a layer (film) constituting the first heater wire 40. FIG. 11B is a fragmentary enlarged view of the surroundings of the first heater wire 40 in FIG. 11A. As is understandable from comparison between FIGS. 11A and 9A, the first heater wire 40 has a planar shape similar to that of the second heater wire 50.

That is, as is understandable from comparison between FIGS. 11A and 1, the first heater wire 40 is disposed at a location overlapping the opening 22 of the substrate 20 in plan view, and the first heater terminal portion 46 is provided above the substrate upper surface 24 of the substrate 20.

The first heater lead-out portion 44 connects the first heater wire 40 and the first heater terminal portion 46. A part of the first heater lead-out portion 44 is disposed at a location overlapping the opening 22 in plan view and is in between the first insulating film 31 and the second insulating film 32. Another part of the first heater lead-out portion 44 is in between the first insulating film peripheral portion 36 (see FIG. 12A) and the second insulating film peripheral portion 37 (see FIG. 10A) above the substrate upper surface 24. The discontinuous portion 45 is provided above the second heater terminal portion 56 (see FIG. 9A) and connects a surface electrode portion 72 (see FIG. 1) exposed to a surface and the second heater terminal portion 56 (see FIG. 3).

As shown in FIG. 5, the flat portion 40b of the first heater wire 40 and the flat portion 50b of the second heater wire 50 are in direct contact, and the first heater wire 40 and the second heater wire 50 are electrically connected in series. That is, as shown in FIGS. 10A and 10B, the second insulating film 32 in between the first heater wire 40 and the second heater wire 50 has the heater connection hole 32j. As shown in FIG. 5, although the second insulating film 32 insulates a heat-generating portion 40a of the first heater wire 40 from the heat-generating portion 50a of the second heater wire 50, the first heater wire 40 and the second heater wire 50 are in contact at their flat portions 40b and 50b via the heater connection hole 32j. Thus, a potential difference is generated between the first heater terminal portion 46 and the second heater terminal portion 56 shown in FIG. 1 to pass a current through the first heater wire 40 and the second heater wire 50 connected in series.

The first heater wire 40 shown in FIG. 11B has larger resistance than that of the second heater wire 50 shown in FIG. 9B. That is, because the first heater wire 40 is thinner than the second heater wire 50 as shown in FIG. 5, the heat-generating portion 40a of the first heater wire 40 has a smaller sectional area orthogonal to the conduction direction than the sectional area of the heat-generating portion 50a of the second heater wire 50. When a current passes through such first heater wire 40 and second heater wire 50, more heat is generated by the first heater wire 40 than by the second heater wire 50.

As shown in FIG. 5, the insulating film 30 having the first heater wire 40 and the second heater wire 50 interposed is substantially flat with respect to the first heater wire 40 and the second heater wire 50 when they are de-energized. By contrast, as the first heater wire 40 and the second heater wire 50 start to be energized during gas detection, the insulating film 30, the first heater wire 40, and the second heater wire 50 thermally expand according to the amount of heat generated. At this time, the amount of thermal expansion of the first heater wire 40 and its surroundings, which are at a relatively upper side, is larger than the amount of thermal expansion of the second heater wire 50 and its surroundings, which are at a relatively lower side. Thus, as shown in FIG. 6, the insulating film 30 warps so as to protrude upwards during gas detection.

Note that a method of making the first heater wire 40 have larger resistance than that of the second heater wire 50 is not limited to making the first heater wire 40 and the second heater wire 50 have different thicknesses. Other such methods may include, for example, making the heat- generating portions 40a and 50a of the first heater wire 40 and the second heater wire 50 have different widths in the film surface direction D2 or different wiring lengths and making the first heater wire 40 and the second heater wire 50 from different materials.

The first heater wire 40, the first heater lead-out portion 44, the first heater terminal portion 46, the discontinuous portion 45, the second heater wire 50, the second heater lead-out portion 54, and the second heater terminal portion 56 may be made from any conductive material. In terms of enabling a high-temperature process in a step of forming the gas detecting unit 60, the conductive material may have a relatively high melting point. Examples of such materials include molybdenum (Mo), platinum (Pt), nickel (Ni), chromium (Cr), tungsten (W), tantalum (Ta), palladium (Pd), iridium (Ir), and an alloy containing two or more of these elements (e.g., a nickel-chromium alloy (NiCr)). The first heater wire 40 and the second heater wire 50 may be made from the same material or from different materials.

### 1.5. Gas Detecting Unit

As shown in FIG. 2, the gas detecting unit 60 is provided on the upper surface of the insulating film 30. As shown in FIG. 5, in the gas sensor 10 having the insulating film 30, which is the multilayer including the first to third insulating films 31, 32, and 33, the gas detecting unit 60 is provided on an upper surface of the first insulating film 31 above the first heater wire 40. The gas detecting unit 60 includes the thermistor film 61 and the electrode portions 62a and 62b, which detect changes in resistance of the thermistor film 61.

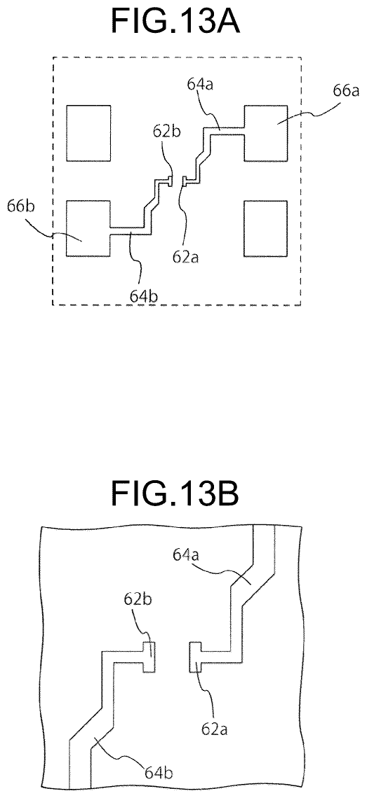

FIG. 13A is a plan view of the electrode portions 62a and 62b, and electrode lead-out portions 64a and 64b and the electrode terminal portions 66a and 66b, which are parts of a film constituting the electrode portions 62a and 62b. FIG. 13B is a fragmentary enlarged view of the surroundings of the electrode portions 62a and 62b in FIG. 13A.

As shown in FIG. 13A, the electrode terminal portions 66a and 66b are disposed above the substrate upper surface 24 of the substrate 20. The electrode lead-out portion 64a connects the electrode portion 62a and the electrode terminal portion 66a provided on the first insulating film 31. The electrode lead-out portion 64b connects the electrode portion 62b and the electrode terminal portion 66b provided on the first insulating film 31. As shown in FIG. 13B, the electrode portions 62a and 62b have a predetermined distance therebetween on the first insulating film 31.

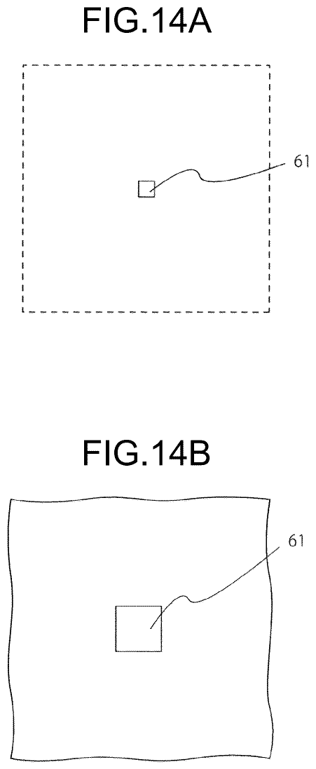

FIG. 14A is a plan view of a planar shape of the thermistor film 61 and its arrangement above the substrate 20. FIG. 14B is a fragmentary enlarged view of the surroundings of the thermistor film 61 in FIG. 14A. The thermistor film 61 is provided on the insulating film 30, particularly on the first insulating film 31 as shown in FIG. 5 at the central portion of the first insulating film 31 so as to connect the electrode portion 62a and the electrode portion 62b. As shown in FIGS. 1 and 5, the thermistor film 61 and the flat portions 40b and 50b of the first heater wire 40 and the second heater wire 50 are disposed so as to at least partly overlap when viewed from above.

During gas detection, in which the gas detecting unit 60 can detect a gas, the first heater wire 40 and the second heater wire 50 are energized to generate heat. This heats the gas detecting unit 60 to reach a temperature at which the gas can suitably be detected. Examples of materials of the thermistor film 61 of the gas detecting unit 60 include CoNiMnOx. Other materials whose resistance changes at the time of contact with a specific gas may also be used. Examples of materials of the electrode portions 62a and 62b, the electrode lead-out portions 64a and 64b, and the electrode terminal portions 66a and 66b include conductive materials, such as platinum (Pt), silver (Ag), gold (Au), and aluminum (Al).

Note that, as shown in FIGS. 1 and 3, uppermost layers of the electrode terminal portions 66a and 66b and the first and second heater terminal portions 46 and 56 are provided with surface electrode portions 71, 72, 73, and 74. Examples of materials of the surface electrode portions 71, 72, 73, and 74 include conductive materials, such as platinum (Pt), silver (Ag), gold (Au), and aluminum (Al).

### 1.6. Manufacturing Method

An example method of manufacturing the gas sensor 10 is described below. However, methods of manufacturing the gas sensor 10 are not limited to the following method. To manufacture the gas sensor 10, a base material (raw material) of the substrate 20 is prepared first. The base material is in a plate shape without the opening 22. Then, the third insulating film 33, the third insulating film peripheral portion 38, and the like (see FIG. 8A) are formed on one main surface, which is the substrate upper surface 24, of the prepared base material. As a method of forming the third insulating film 33 and the third insulating film peripheral portion 38, a known film formation method (e.g., a thermal oxidation method or a chemical vapor deposition (CVD) method) is used.

Then, on the third insulating film 33, the second heater wire 50 is formed. In the step of forming the second heater wire 50, the second heater lead-out portion 54 and the second heater terminal portion 56, which constitute the same film as that of the second heater wire 50, are also formed at the same time (see FIG. 9A). Shapes of the second heater wire 50, the second heater lead-out portion 54, and the second heater terminal portion 56 are formed using, for example, lift-off. In the lift-off step, first, a resist is applied to an entire surface, where a predetermined pattern is to be formed, and is exposed to light for development into the shape of the predetermined pattern. Development dissolves the resist corresponding to the shape of the predetermined pattern for patterning. After the resist is dissolved, a material for forming the pattern is deposited using a film formation method (e.g., sputtering or vapor deposition). After film formation, the remaining resist is removed using a resist stripper. This also removes the material deposited on the resist and leaves the deposited material only in the patterned region, forming the predetermined pattern.

Then, on the second heater wire 50, the second insulating film 32 is formed. In the step of forming the second insulating film 32, the second insulating film peripheral portion 37, which constitutes the same film as that of the second insulating film 32, is also formed at the same time (see FIG. 10A). In the step of forming the second insulating film 32 and the second insulating film peripheral portion 37, a known film formation method or the like is used, similarly to formation of the third insulating film 33.

Then, on the second insulating film 32, the first heater wire 40 is formed. In the step of forming the first heater wire 40, the first heater lead-out portion 44, the first heater terminal portion 46, and the discontinuous portion 45, which constitute the same film as that of the first heater wire 40, are also formed at the same time (see FIG. 11A). A method of forming the first heater wire 40 and the like is similar to the method of forming the second heater wire 50 described before.

Then, on the first heater wire 40, the first insulating film 31 is formed. In the step of forming the first insulating film 31, the first insulating film peripheral portion 36, which constitutes the same film as that of the first insulating film 31, is also formed at the same time (see FIG. 12A). In the step of forming the first insulating film 31 and the first insulating film peripheral portion 36, a known film formation method or the like can be used, similarly to formation of the third insulating film 33 and the second insulating film 32.

Then, on the first insulating film 31, the gas detecting unit 60 is formed. In the step of forming the gas detecting unit 60, the electrode portions 62a and 62b, the electrode lead-out portions 64a and 64b, and the electrode terminal portions 66a and 66b shown in FIG. 13A are formed at once; and then the thermistor film 61 (see FIG. 14A) is formed on the electrode portions 62a and 62b. Similarly to the first and second heater wires 40 and 50, shapes of the electrode portions 62a and 62b and the like are formed using, for example, lift-off. Further, above the first and second heater terminal portions 46 and 56 and the electrode terminal portions 66a and 66b, the surface electrode portions 71 to 74 are formed. Note that the thermistor film 61 may be formed on the electrode portions 62a and 62b after the step of forming the surface electrode portions 71 to 74 or a step of forming the opening 22 described later.

Then, an etching mask is applied to a predetermined region of a main surface where the third insulating film 33, the third insulating film peripheral portion 38, and the like are not formed among the main surfaces of the base material; and the base material is etched until the third insulating film 33 and the like formed on the opposite main surface (substrate upper surface 24) are exposed. This forms the opening 22. The gas sensor 10, in which the insulating film 30 is disposed at the location overlapping the opening 22 in plan view as shown in FIG. 2, is thus manufactured.

### 1.7. Operation

In the gas sensor 10, the first heater wire 40 and the second heater wire 50 are connected to an external circuit not shown in the drawings via the first and second heater lead-out portions 44 and 54, the first and second heater terminal portions 46 and 56, the surface electrode portions 71 and 72, and the like. Also, the electrode portions 62a and 62b of the gas detecting unit 60 are connected to an external circuit not shown in the drawings via the electrode lead-out portions 64a and 64b, the electrode terminal portions 66a and 66b, the surface electrode portions 73 and 74, and the like. Once the gas sensor 10 is started, the first and second heater wires 40 and 50 start to be energized by a control unit and the external circuit, and the gas detecting unit 60 and its surroundings are heated to a predetermined temperature. The gas detecting unit 60 thus goes into a gas detectable state.

As shown in FIG. 5, the insulating film 30 having the first heater wire 40 and the second heater wire 50 interposed is substantially flat with respect to the first heater wire 40 and the second heater wire 50 when they are de-energized. However, after the first and second heater wires 40 and 50 start to be energized, during gas detection, in which the gas detecting unit 60 can detect a gas, the insulating film 30 warps so as to protrude upwards as shown in FIG. 6. This is because the resistance of the first heater wire 40 is larger than the resistance of the second heater wire 50 connected to the first heater wire 40 in series, making the first heater wire 40 generate more heat than the second heater wire 50.

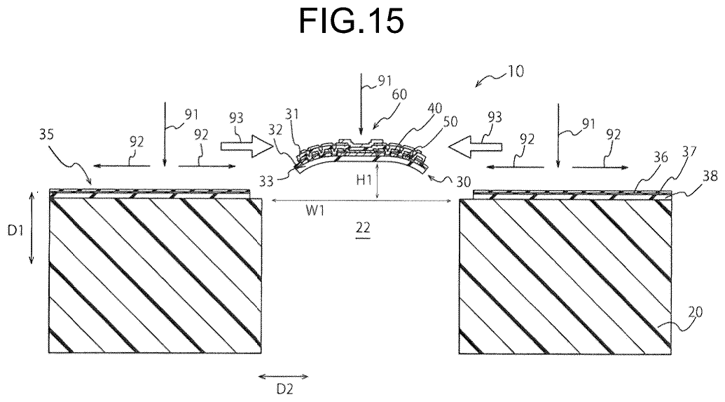

FIG. 15 is a schematic view of a shape of the gas sensor 10 during gas detection and a gas flow near the gas sensor 10. As shown in FIG. 15, during gas detection, a gas may flow in the vertical direction D1 as shown by arrows 91 or in the film surface direction D2 as shown by arrows 92 or arrows 93. If the insulating film 30 of the gas sensor 10 is flat during gas detection, the gas detecting unit 60 of the gas sensor 10 has less chances of detecting the gas flow in the film surface direction D2 shown by the arrows 92 or 93. This may cause delays in detection output.

However, the insulation film 30 of the gas sensor 10 actually warps so as to protrude upwards during gas detection as shown in FIG. 15. Thus, in the gas sensor 10, the gas detecting unit 60 has more chances of detecting the gas flow in the film surface direction D2 shown by the arrows 92 or 93 compared to a situation where the insulating film 30 remains flat. The gas sensor 10 thus has high high-speed responsivity to the gas flow in the film surface direction D2. Note that, whether the insulating film 30 remains flat or not, the gas sensor 10 has the same responsiveness to the gas flow in the vertical direction DI shown by the arrows 91.

That is, because the insulating film, where the gas detecting unit is installed, of such a gas sensor warps upwards, the gas detecting unit can have better chances of coming into contact with a gas flow approaching in a horizontal direction with respect to the gas detecting unit. Consequently, such a gas sensor has suitable responsiveness.

In such a gas sensor, the amount of heat generated by the first heater wire can be larger than the amount of heat generated by the second heater wire to deform the insulating film into a warped shape protruding upwards using the heat generated by these heater wires. Because the heat for heating the gas detecting unit is repurposed for deformation of the insulating film into the warped shape in such a gas sensor, energy is efficiently utilized.

In such a gas sensor, a predetermined relationship between the amounts of expansion of the first heater wire and the second heater wire in the film surface direction can deform the insulating film into the warped shape protruding upwards in response to deformation of these heater wires when they are heated. Because the heat for heating the gas detecting unit is repurposed for deformation of the insulating film into the warped shape in such a gas sensor, energy is efficiently utilized.

### 2. Second Embodiment

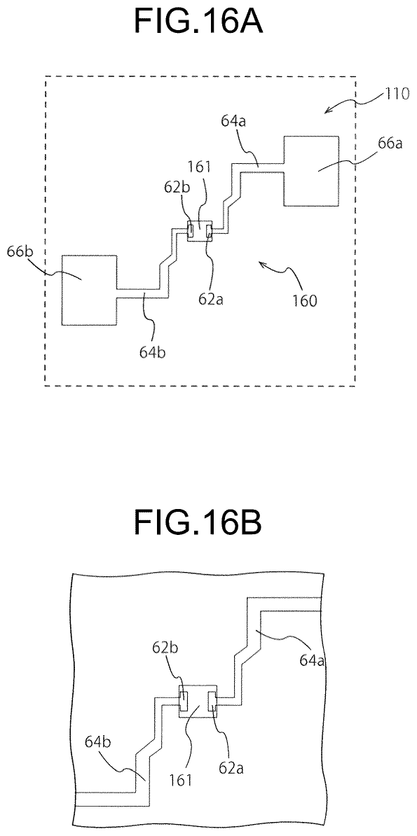

FIG. 16A is a plan view of a shape of a gas detecting unit 160 of a gas sensor 110 according to a second embodiment of the present disclosure. FIG. 16B is a fragmentary enlarged view of the surroundings of a MOx material film 161 shown in FIG. 16A. The gas sensor 110 is different from the gas sensor 10 according to the first embodiment in that the gas detecting unit 160 includes the MOx material film 161, which is a metal oxide film, instead of the thermistor film 61; however, the gas sensor 110 is similar to the gas sensor 10 shown in FIG. 1 in other respects. Note that contact of the gas detecting unit 160 with a gas of interest causes a redox reaction to change resistance of the metal oxide film. Examples of gases detectable by the gas detecting unit 160 include flammable gases and reducing gases, such as, for example, carbon monoxide (CO), methane (CH₄), propane (C₃H₈), ethanol (C₂H₅OH), and hydrogen (H₂). Examples of the MOx material film 161 (metal oxide film) include tin oxide (SnO₂), zirconium oxide (ZrO₂), iron oxide (Fe₂O₃), tungsten oxide (WO₃), indium oxide (In₂O₃), and cobalt oxide (CO₃O₄). Noble metal particles (e.g., platinum (Pt) or palladium (Pd)) may be supported on the MOx material film 161.

The MOx material film 161 shown in FIG. 16A is provided on an upper surface of an insulating film 30 (see FIG. 5), similarly to the thermistor film 61 shown in FIG. 14A. In particular, as shown in FIGS. 5 and 16A, the MOx material film 161 is provided at a central portion of a first insulating film 31 so as to connect an electrode portion 62a and an electrode portion 62b on the first insulating film 31.

The gas sensor 110 includes a first heater wire 40 and a second heater wire 50, which are provided inside the insulating film 30 (multilayer), similarly to the gas sensor 10 shown in FIG. 1. In the gas sensor 110, the first heater wire 40 and the second heater wire 50 are energized to generate heat during gas detection, similarly to the gas sensor 10. This heats the gas detecting unit 160 to reach a temperature at which a gas can suitably be detected.

In the gas sensor 110, the insulating film 30 warps so as to protrude upwards during gas detection similarly to the gas sensor 10 shown in FIG. 15; and the speed of response of the gas sensor 110 during detection of a gas of interest is fast. Other than that, what is common to the gas sensor 110 shown in FIGS. 16A and 16B and the gas sensor 10 shown in FIG. 1 and the like exhibits similar effects brought by the gas sensor 10.

### 3. Third Embodiment

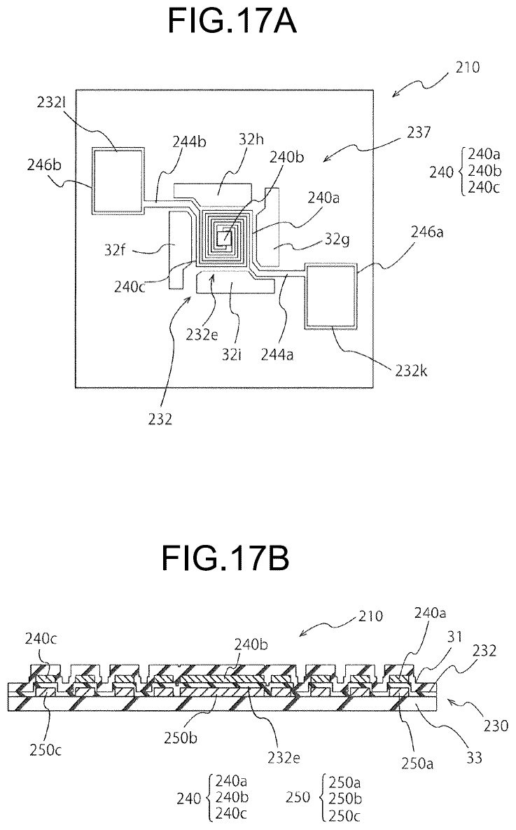

FIG. 17A is a plan view of planar shapes of a first heater wire 240, first heater lead-out portions 244a and 244b, and first heater terminal portions 246a and 246b; and a second insulating film 232 and a second insulating film peripheral portion 237 of a gas sensor 210 according to a third embodiment of the present disclosure. FIG. 17B is a sectional view of a first insulating film 31, the second insulating film 232, a third insulating film 33, the first heater wire 240, and a second heater wire 250 of the gas sensor 210. Note that FIG. 17A shows members with their outlines overlapping so that the shape of the second insulating film 232 underneath is visible. In FIGS. 17A and 17B, illustrations of a gas detecting unit, a substrate, and the like of the gas sensor 210 are omitted.

The gas sensor 210 according to the third embodiment is different from the gas sensor 10 in that the first heater wire 240 and the second heater wire 250 are electrically connected in parallel and that the first heater wire 240 has smaller resistance than that of the second heater wire 250. However, the gas sensor 210 is similar to the gas sensor 10 according to the first embodiment other than the type of connection between the first heater wire 240 and the second heater wire 250. Description of the gas sensor 210 is provided focusing on the difference between the gas sensors 210 and 10. Description of similarities between the gas sensors 210 and 10 is omitted.

An insulating film 230 of the gas sensor 210 is a multilayer including the first insulating film 31, the second insulating film 232 below the first insulating film 31, and the third insulating film 33 below the second insulating film 232. The first insulating film 31 and the third insulating film 33 have planar shapes similar to those of the gas sensor 10. Although unshown in FIG. 17A, a first insulating film peripheral portion 36, which constitutes the same film as that of the first insulating film 31, and a third insulating film peripheral portion 38, which constitutes the same film as that of the third insulating film 33, have planar shapes similar to those of the gas sensor 10.

By contrast, the second insulating film 232 of the gas sensor 210 shown in FIG. 17A does not have a heater connection hole 32j at a central portion of the second insulating film 232, unlike the second insulating film 32 shown in FIG. 10A. That is, the central portion 232e of the second insulating film 232 does not have a through-hole as shown in FIG. 17B; and the second insulating film 232 electrically insulates the first heater wire 240 on an upper side from the second heater wire 250 on a lower side.

As shown in FIG. 17A, the second insulating film peripheral portion 237, which constitutes the same film as that of the second insulating film 232, has heater insertion holes 232l and 232k. As described later, the first heater wire 240 is drawn out from a central portion of an opening 22 (see FIG. 1) to the two first heater terminal portions 246a and 246b provided at two locations above a substrate upper surface 24. The second insulating film peripheral portion 237 has the two heater insertion holes 232l and 232k corresponding to the two first heater terminal portions 246a and 246b.

The first heater wire 240 shown in FIGS. 17A and 17B is provided in between the first insulating film 31 and the second insulating film 232, similarly to the first heater wire 40 shown in FIGS. 11A and 5. Viewed from above, the first heater wire 240 is disposed at the opening 22 of the substrate 20, and the first heater terminal portions 246a and 246b are provided above the substrate upper surface 24 of the substrate 20. The first heater wire 240 includes a flat portion 240b, which is disposed centrally, and heat-generating portions 240a and 240c in a spiral shape around the flat portion 240b.

With the flat portion 240b at the center, the heat-generating portions 240a and 240c present two spiral planar shapes that do not overlap when viewed from above. An outer end portion of the heat-generating portion 240a is connected to the first heater lead-out portion 244a at the lower right of FIG. 17A to connect to the first heater terminal portion 246a via the first heater lead-out portion 244a. By contrast, an outer end portion of the heat-generating portion 240c is connected to the first heater lead-out portion 244b at the upper left of FIG. 17A to connect to the first heater terminal portion 246b via the first heater lead-out portion 244b.

The second heater wire 250 shown in FIG. 17B is provided in between the second insulating film 232 and the third insulating film 33, similarly to the second heater wire 50 shown in FIGS. 9A and 5. Although unshown in FIG. 17A, the second heater wire 250 has a planar shape similar to that of the first heater wire 240 and includes a flat portion 250b, which is disposed centrally, and heat-generating portions 250a and 250c in a spiral shape around the flat portion 250b. Similarly to the first heater wire 240, the second heater wire is connected to two second heater terminal portions, which are disposed below the first heater terminal portions 246a and 246b, via two second heater lead-out portions.

As described above, the second insulating film 232 in between the first heater wire 240 and the second heater wire 250 does not have a heater connection hole. Thus, the first heater wire 240 and the second heater wire 250 are electrically insulated inside the insulating film 230 shown in FIG. 17B.

However, the first heater terminal portion 246a shown in FIG. 17A is electrically connected to the second heater terminal portion disposed thereunder; and the first heater terminal portion 246b is electrically connected to the second heater terminal portion disposed thereunder. In this manner, the first heater wire 240 and the second heater wire 250 of the gas sensor 210 are electrically connected in parallel.

Unlike the first heater wire 40 and the second heater wire 50 of the gas sensor 10 shown in FIG. 5, the first heater wire 240 of the gas sensor 210 shown in FIG. 17B has smaller resistance than that of the second heater wire 250 thereof. When a current passes through such first heater wire 240 and second heater wire 250, more heat is generated by the first heater wire 240 than by the second heater wire 250.

Similarly to the gas sensor 10 shown in FIG. 5, the gas detecting unit of the gas sensor 210 is provided on an upper surface of the first insulating film 31. Thus, similarly to the gas sensor 10, the insulating film 230 of the gas sensor 210 warps so as to protrude upwards like the insulating film 30 shown in FIG. 6 does during gas detection, in which the gas detecting unit can detect a gas. This increases chances of detecting a gas of interest. Other than that, what is common to the gas sensor 210 shown in FIGS. 17A and 17B and the gas sensor 10 shown in FIG. 1 and the like exhibits similar effects brought by the gas sensor 10.

That is, in such a gas sensor, the amount of heat generated by the first heater wire can be larger than the amount of heat generated by the second heater wire to deform the insulating film into a warped shape protruding upwards using the heat generated by these heater wires. Because the heat for heating the gas detecting unit is repurposed for deformation of the insulating film into the warped shape in such a gas sensor, energy is efficiently utilized.

### 4. Fourth Embodiment

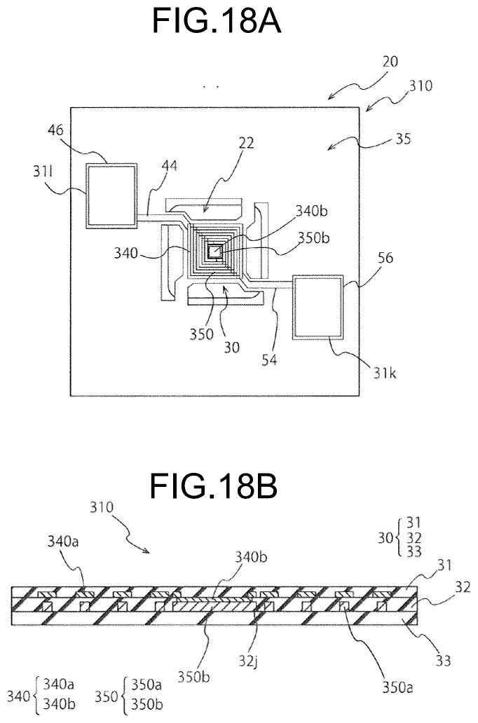

FIG. 18A is a plan view of planar shapes of a substrate 20, an insulating film 30, an insulating film peripheral portion 35, a first heater wire 340, a first heater lead-out portion 44, a first heater terminal portion 46, a second heater wire 350, a second heater lead-out portion 54, and a second heater terminal portion 56 of a gas sensor 310 according to a fourth embodiment of the present disclosure. FIG. 18B is a sectional view of a first insulating film 31, a second insulating film 32, a third insulating film 33, the first heater wire 340, and the second heater wire 350 of the gas sensor 310. Note that FIG. 18A shows only the substrate 20 and the third insulating film 33 in a nontransparent manner and other members with their outlines overlapping so that shapes of members underneath are visible. In FIGS. 18A and 18B, illustrations of a gas detecting unit and the like of the gas sensor 310 are omitted.

As shown in FIG. 18B, the insulating film 30 of the gas sensor 310 is a multilayer including the first insulating film 31, the second insulating film 32 below the first insulating film 31, and the third insulating film 33 below the second insulating film 32 and has a planar shape similar to that of the gas sensor 10. However, upper surfaces of the first insulating film 31 and the second insulating film 32 of the gas sensor 310 are flattened so that the upper surfaces of these films have no bumps or dents tracing bumps or dents of lower surfaces of these films. Note that, in the gas sensor of each embodiment, whether to flatten the upper surfaces of the first insulating film 31 and the second insulating film 32 is optional. The upper surfaces of the first insulating film 31 and the second insulating film 32 of the gas sensor 10 may be flattened. It may be that the upper surfaces of the first insulating film 31 and the second insulating film 32 of the gas sensor 310 are not flattened. Description of the gas sensor 310 is provided focusing on the difference between the gas sensors 310 and 10. Description of similarities between the gas sensors 310 and 10 is omitted.

The first heater wire 340 shown in FIGS. 18A and 18B is provided in between the first insulating film 31 and the second insulating film 32, similarly to the first heater wire 40 shown in FIGS. 11A and 5. Viewed from above, the first heater wire 340 is disposed at an opening 22 of the substrate 20, and the first heater terminal portion 46 is provided above a substrate upper surface 24 of the substrate 20. The first heater wire 340 includes a flat portion 340b, which is disposed centrally, and a heat-generating portion 340a in a spiral shape around the flat portion 340b.

The second heater wire 350 is provided in between the second insulating film 32 and the third insulating film 33, similarly to the second heater wire 50 shown in FIGS. 9A and 5. Viewed from above, the second heater wire 350 is disposed at the opening 22 of the substrate 20, and the second heater terminal portion 56 is provided above the substrate upper surface 24 of the substrate 20. The second heater wire 350 includes a flat portion 350b, which is disposed centrally, and a heat-generating portion 350a in a spiral shape around the flat portion 350b.

As shown in FIG. 18B, the flat portion 340b of the first heater wire 340 and the flat portion 350b of the second heater wire 350 are in direct contact, and the first heater wire 340 and the second heater wire 350 are electrically connected in series. That is, the second insulating film 32 in between the first heater wire 340 and the second heater wire 350 has a heater connection hole 32j, through which the flat portion 340b of the first heater wire 340 and the flat portion 350b of the second heater wire 350 are connected.

As shown in FIG. 18B, the heat-generating portion 340a of the first heater wire 340 and the heat-generating portion 350a of the second heater wire 350 have different sectional shapes in a section that passes a center of the opening 22 and is perpendicular to the substrate upper surface 24. That is, the heat-generating portion 340a of the first heater wire 340 has a smaller thickness in the vertical direction D1 and a larger width in the film surface direction D2 than those of the heat-generating portion 350a of the second heater wire 350.

In such a gas sensor 310, the first heater wire 340 has a larger amount of expansion in the film surface direction D2 during heating with respect to an unheated state than that of the second heater wire 350. This is because the amounts of expansion of the heat-generating portions 340a and 350a in the film surface direction D2 are in proportion to their widths in the film surface direction D2.

Although unshown in FIGS. 18A and 18B, the gas detecting unit of the gas sensor 310 is provided on an upper surface of the first insulating film 31, similarly to the gas detecting unit 60 of the gas sensor 10 shown in FIG. 2 and the like. As the first heater wire 340 and the second heater wire 350 are energized to generate heat during gas detection by the gas sensor 310, the difference in the amounts of expansion described above makes the insulating film 30 of the gas sensor 310 warp so as to protrude upwards (see FIGS. 6 and 15).

Thus, in the gas sensor 310, the gas detecting unit has more chances of detecting a gas flow in the film surface direction D2, and the speed of response (output) increases. Even if the first heater wire 340 and the second heater wire 350 of the gas sensor 310 have the same resistance, a difference in their aspect ratios, which are ratios of the thicknesses of the heat-generating portions 340a and 350a in the vertical direction D1 to the widths thereof in the film surface direction D2, can deform the insulating film 30 into a warped shape protruding upwards. Other than that, what is common to the gas sensor 310 shown in FIGS. 18A and 18B and the gas sensor 10 shown in FIG. 1 and the like exhibits similar effects brought by the gas sensor 10.

### 5. Fifth Embodiment

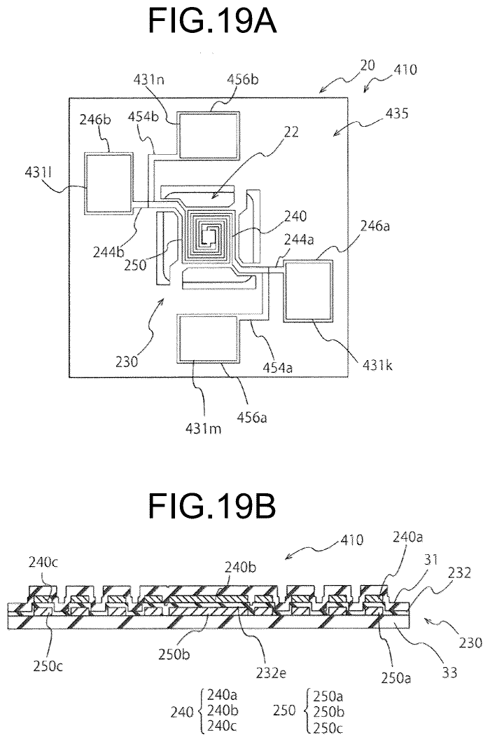

FIG. 19A is a plan view of planar shapes of a substrate 20, an insulating film 230, an insulating film peripheral portion 435, a first heater wire 240, first heater lead-out portions 244a and 244b, first heater terminal portions 246a and 246b, a second heater wire 250, second heater lead-out portions 454a and 454b, and second heater terminal portions 456a and 456b of a gas sensor 410 according to a fifth embodiment of the present disclosure. FIG. 19B is a sectional view of a first insulating film 31, a second insulating film 232, a third insulating film 33, the first heater wire 240, and the second heater wire 250 of the gas sensor 410. Note that FIG. 19A shows only the substrate 20 and the third insulating film 33 in a nontransparent manner and other members with their outlines overlapping so that shapes of members underneath are visible. In FIGS. 19A and 19B, illustrations of a gas detecting unit and the like of the gas sensor 410 are omitted. As is understandable from comparison between FIGS. 19B and 17B, the insulating film 230, the first heater wire 240, and the second heater wire 250 of the gas sensor 410 have shapes similar to those of the gas sensor 210 according to the third embodiment. However, the gas sensor 410 is different from the gas sensor 210 in that the second heater wire 250 of the gas sensor 410 is connected to the second heater terminal portions 456a and 456b provided independently from the first heater terminal portions 246a and 246b, to which the first heater wire 240 is connected. Description of the gas sensor 410 is provided focusing on the difference between the gas sensors 410 and 210. Description of similarities between the gas sensors 410 and 210 is omitted.

As shown in FIG. 19B, the insulating film 230 of the gas sensor 410 is a multilayer including the first insulating film 31, the second insulating film 232 below the first insulating film 31, and the third insulating film 33 below the second insulating film 232. A central portion 232e of the second insulating film 232 of the gas sensor 410 does not have a through-hole, similarly to the second insulating film 232 shown in FIG. 17A; and the second insulating film 232 electrically insulates the first heater wire 240 on an upper side from the second heater wire 250 on a lower side.

By contrast, as shown in FIG. 19A, in the gas sensor 410, locations of the first heater terminal portions 246a and 246b do not overlap locations of the second heater terminal portions 456a and 456b viewed from above. The second heater lead-out portion 454a, which connects a heat-generating portion 250a and the second heater terminal portion 456a, has a shape different from that of the first heater lead-out portion 244a. The second heater lead-out portion 454b, which connects a heat-generating portion 250c and the second heater terminal portion 456b, has a shape different from that of the first heater lead-out portion 244b.

As shown in FIG. 19A, a first insulating film peripheral portion and a second insulating film peripheral portion of the insulating film peripheral portion 435 provided above the second heater terminal portions 456a and 456b have heater insertion holes 431m and 43 In and the like, which allow electrical connection to the second heater terminal portions 456a and 456b. In the gas sensor 410, the first heater wire 240 and the first heater lead-out portions 244a and 244b and the first heater terminal portions 246a and 246b connected thereto are insulated from the second heater wire 250 and the second heater lead-out portions 454a and 454b and the second heater terminal portions 456a and 456b connected thereto. Thus, the first heater wire 240 and the second heater wire 250 are electrically independent.

In the gas sensor 410 shown in FIG. 19A, the first heater wire 240 and the second heater wire 250 are independently connected to a control unit and an external circuit via the first heater terminal portions 246a and 246b or the second heater terminal portions 456a and 456b. Thus, a current or a voltage applied to the first heater wire 240 and a current or a voltage applied to the second heater wire 250 can be independently controlled using, for example, independent control signals transmitted from the control unit.

Even if the first heater wire 240 and the second heater wire 250 of such a gas sensor 410 are made from the same material and have the same shape, the amounts of heat generation of the first heater wire 240 and the second heater wire 250 can be controlled. Thus, in the gas sensor 410, current values or the like can be controlled so that the amount of heat generated by the first heater wire 240 is larger than the amount of heat generated by the second heater wire 250. This can deform the insulating film 230 into a warped shape protruding upwards as shown in FIG. 6. This can improve the speed of response of the gas sensor 410 during gas detection. Other than that, what is common to the gas sensor 410 shown in FIGS. 19A and 19B and the gas sensor 210 shown in FIGS. 17A and 17B and the like exhibits similar effects brought by the gas sensor 210.

In such a gas sensor, independently controlling currents, voltages, or the like applied to the first heater wire and the second heater wire can deform the insulating film into a warped shape protruding upwards using the heat generated by these heater wires. Because the heat for heating the gas detecting unit is repurposed for deformation of the insulating film into the warped shape in such a gas sensor, energy is efficiently utilized.

Hereinabove, the gas sensors according to the present disclosure have been described with reference to the embodiments; however, needless to say, gas sensors according to the present disclosure are not limited to these embodiments and include many other embodiments and modified examples. For example, an insulating film of a gas sensor is not limited to a three-layered multilayer shown in the embodiments and may be a multilayer including two layers or including four or more layers. Also, the orientation in which the gas sensor is installed is optional. The vertical direction D1 of the gas sensor at the time of installation of the gas sensor and the gravity direction may be different or may be the same.

The gas sensors according to the present disclosure are not limited to those whose insulating film warps upwards at the time of energization of the heaters as in the first to fifth embodiments. Embodiments with an insulating film already having a warped shape protruding upwards prior to energization of heaters are conceivable. For example, setting a formation temperature (substrate temperature at the time of film deposition) of a first insulating film 31, which is on an upper side, higher than a formation temperature of a third insulating film 33, which is on a lower side, generates stress for the third insulating film 33 and the first insulating film 31 to warp upwards. This allows the insulating film to be warped so as to protrude upwards prior to energization of heaters. Note that, because such stress to the insulating film is supported by a substrate 20 not having an opening 22 yet, no warps of the third insulating film 33 or the first insulating film 31 are generated at a manufacturing stage prior to formation of the opening 22; and no obstacles are posed in steps of forming heater wires or a gas detecting unit. Also, in such a gas sensor, a heater wire can have a one-layer structure including a second heater wire 50 to omit a first heater wire 40, and the insulating film can have a two-layer structure including the first and third insulating films 31 and 33 to omit a second insulating film 32.

Other embodiments with an insulating film that warps so as to protrude upwards at the time of energization of heaters include an embodiment in which a first insulating film 31, which is on an upper side, has a higher film density than that of a third insulating film 33, which is on a lower side. The film densities of the first and third insulating films 31 and 33 can be controlled by, for example, making the first and third insulating films 31 and 33 take different amounts of a gas component using different pressures of a gas at the time of formation of the first and third insulating films 31 and 33. For example, relatively decreasing the gas pressure of film formation relatively increases the film density, whereas relatively increasing the gas pressure of film formation relatively decreases the film density. In such a gas sensor, a heater wire can have a one-layer structure including a second heater wire 50 to omit a first heater wire 40, and the insulating film can have a two-layer structure including the first and third insulating films 31 and 33 to omit a second insulating film 32.

Also, in the gas sensors according to the present disclosure, how much the insulating film 30 warps so as to protrude upwards is not limited; the amount of upward protrusion (protrusion height H1) of the insulating film 30 during gas detection may be, for example, 1% or more with respect to a maximum width W1 in the film surface direction D2 of the insulating film 30 overlapping the opening 22 in plan view (see FIG. 15).

As is understood from the above description, the present specification discloses the following.
[1]

A gas sensor including:
a substrate with an opening;
an insulating film disposed at a location overlapping the opening in plan view; and
a gas detecting unit on an upper surface of the insulating film,
in which the insulating film is configured to have a warped shape protruding upwards during gas detection, in which the gas detecting unit can detect a gas.
[2]

The gas sensor according to [1], in which
the insulating film includes a multilayer including a first insulating film, a second insulating film below the first insulating film, and a third insulating film below the second insulating film;
the gas detecting unit is on an upper surface of the first insulating film;
the gas sensor includes a first heater wire in between the first insulating film and the second insulating film and a second heater wire in between the second insulating film and the third insulating film;
the first heater wire and the second heater wire are electrically connected in series; and the first heater wire is configured to have larger resistance than that of the second heater wire.
[3]

The gas sensor according to [1], in which the insulating film includes a multilayer including a first insulating film, a second insulating film below the first insulating film, and a third insulating film below the second insulating film;
the gas detecting unit is on an upper surface of the first insulating film;
the gas sensor includes a first heater wire in between the first insulating film and the second insulating film and a second heater wire in between the second insulating film and the third insulating film;
the first heater wire and the second heater wire are electrically connected in parallel; and
the first heater wire is configured to have smaller resistance than that of the second heater wire.
[4]

The gas sensor according to [1] or [2], in which the insulating film includes a multilayer including a first insulating film, a second insulating film below the first insulating film, and a third insulating film below the second insulating film;
the gas detecting unit is on an upper surface of the first insulating film;
the gas sensor includes a first heater wire in between the first insulating film and the second insulating film and a second heater wire in between the second insulating film and the third insulating film; and
the first heater wire is configured to have a larger amount of expansion in a film surface direction during heating with respect to an unheated state than that of the second heater wire.
[5]

The gas sensor according to [1], in which
the insulating film includes a multilayer including a first insulating film, a second insulating film below the first insulating film, and a third insulating film below the second insulating film;
the gas detecting unit is on an upper surface of the first insulating film;
the gas sensor includes a first heater wire in between the first insulating film and the second insulating film and a second heater wire in between the second insulating film and the third insulating film; and
the first heater wire and the second heater wire are electrically independent.

### REFERENCE NUMERALS

10, 110, 210, 310, 410 . . . gas sensor
20 . . . substrate
22 . . . opening
24 . . . substrate upper surface
30, 230 . . . insulating film
31a, 31b, 31c, 31d, 32a, 32b, 32c, 32d, 33a, 33b, 33c, 33d . . . beam portion
33e, 32e, 31e, 232e . . . central portion
31f, 31g, 31h, 31i, 32f, 32g, 32h, 32i, 33f, 33g, 33h, 33i . . . peripheral hole
31 . . . first insulating film
32, 232 . . . second insulating film
33 . . . third insulating film
32j . . . heater connection hole
32k, 31k, 31l, 232k, 232l, 431k, 431k, 431m, 431n . . . heater insertion hole
35, 435 . . . insulating film peripheral portion
36 . . . first insulating film peripheral portion
37, 237 . . . second insulating film peripheral portion
38 . . . third insulating film peripheral portion
40, 240, 340 . . . first heater wire
40a, 50a, 250a, 250c, 240a, 240c, 350a, 340a . . . heat-generating portion
40b, 50b, 250b, 240b, 350b, 340b . . . flat portion
44, 244a, 244b . . . first heater lead-out portion
45 . . . discontinuous portion
46, 246a, 246b . . . first heater terminal portion
50, 250, 350 . . . second heater wire
54, 454a, 454b . . . second heater lead-out portion
56, 456a, 456b . . . second heater terminal portion
60, 160 . . . gas detecting unit
61 . . . thermistor film
62a, 62b . . . electrode portion
64a, 64b . . . electrode lead-out portion
66a, 66b . . . electrode terminal portion
71, 72, 73, 74 . . . surface electrode portion
91, 92, 93 . . . arrow
D1 . . . vertical direction
D2 . . . film surface direction
161. . . . MOx material film

What is claimed is:

1. A gas sensor comprising:
a substrate with an opening;
an insulating film disposed at a location overlapping the opening in plan view; and
a gas detecting unit on an upper surface of the insulating film,
wherein the insulating film is configured to have a warped shape protruding upwards during gas detection, in which the gas detecting unit can detect a gas.

2. The gas sensor according to claim 1, wherein the insulating film comprises a multilayer including a first insulating film, a second insulating film below the first insulating film, and a third insulating film below the second insulating film;
the gas detecting unit is on an upper surface of the first insulating film;
the gas sensor comprises a first heater wire in between the first insulating film and the second insulating film and a second heater wire in between the second insulating film and the third insulating film;
the first heater wire and the second heater wire are electrically connected in series; and
the first heater wire is configured to have larger resistance than that of the second heater wire.

3. The gas sensor according to claim 1, wherein
the insulating film comprises a multilayer including a first insulating film, a second insulating film below the first insulating film, and a third insulating film below the second insulating film;
the gas detecting unit is on an upper surface of the first insulating film;
the gas sensor comprises a first heater wire in between the first insulating film and the second insulating film and a second heater wire in between the second insulating film and the third insulating film;
the first heater wire and the second heater wire are electrically connected in parallel; and
the first heater wire is configured to have smaller resistance than that of the second heater wire.

4. The gas sensor according to claim 1, wherein
the insulating film comprises a multilayer including a first insulating film, a second insulating film below the first insulating film, and a third insulating film below the second insulating film;
the gas detecting unit is on an upper surface of the first insulating film;
the gas sensor comprises a first heater wire in between the first insulating film and the second insulating film and a second heater wire in between the second insulating film and the third insulating film; and
the first heater wire is configured to have a larger amount of expansion in a film surface direction during heating with respect to an unheated state than that of the second heater wire.

5. The gas sensor according to claim 1, wherein
the insulating film comprises a multilayer including a first insulating film, a second insulating film below the first insulating film, and a third insulating film below the second insulating film;
the gas detecting unit is on an upper surface of the first insulating film;
the gas sensor comprises a first heater wire in between the first insulating film and the second insulating film and a second heater wire in between the second insulating film and the third insulating film; and
the first heater wire and the second heater wire are electrically independent.
